(19) United States
(12) Patent Application Publication
(10) Pub. No.: US 2022/0233346 A1
(43) Pub. Date: Jul. 28, 2022

(54) COLD THERAPY GARMENT

(71) Applicant: My Relief Wear, LLC, Taylors, SC (US)

(72) Inventor: Mary A. McElya, Taylors, SC (US)

(73) Assignee: My Relief Wear, LLC, Taylors, SC (US)

(21) Appl. No.: 17/582,353

(22) Filed: Jan. 24, 2022

Related U.S. Application Data
(60) Provisional application No. 63/199,811, filed on Jan. 27, 2021.

(51) Int. Cl. A61F 7/02

(52) U.S. Cl. CPC A61F 7/02; A61F 2007/0008

(57) ABSTRACT

Described herein are cold therapy devices and methods for making same and, more particularly, to a garment comprising cold therapy components that may be placed adjacent a wearer's body at various locations while remaining fixed in place during activities, such as walking or movement, without requiring the wearer to hold the cold components in place or remain sedentary.

### TECHNICAL FIELD

The subject matter disclosed herein is generally directed to a cold therapy device and methods for making same and, more particularly, to a garment comprising cold therapy components that may be placed adjacent a wearer's body at various locations while remaining fixed in place during activities, such as walking or movement, without requiring the wearer to hold the cold components in place or remain sedentary.

### BACKGROUND

We all have aches, sprains, or joint pains that require icing for relief. All currently available cold therapy options have the same issues: they restrict mobility, they are uncomfortable, and they do not stay in place. Cold therapy is usually a humdrum routine in which the user is uncomfortably chained to a seat for thirty minutes to an hour. Further requiring one to chase the ice pack as it slips and then needing to change pants because the pack leaked.

Traditional cold therapy devices are bulky and may be poorly made, including ice packs, water circulation machines, topical creams, cryotherapy, and homemade options like frozen peas. Currently available ice pack clothing is limited and expensive, such as a tactical vest or expensive athlete-focused spandex wear with insertable and removable ice packs. Additionally, casual everyday wear with completely encapsulated cold packs does not exist. The process of having a cold therapy or “ice down” regimen must change. In today's busy world, stopping to “ice-down” is not practical.

Accordingly, it is an object of the present disclosure to provide a cold therapy device that does not require the user to sit or remain sedentary for thirty minutes or more. The current disclosure also eliminates slipping ice packs and the need to throw away or wash a favorite pair of sweats because a pack leaked or was poorly made and stained the wearer's clothing.

Citation or identification of any document in this application is not an admission that such a document is available as prior art to the present disclosure.

### SUMMARY

The above objectives are accomplished according to the present disclosure by providing in one embodiment a cooling garment. The garment may include a garment body, at least one panel affixed to the garment body, the panel configured to conform to at least one part of a wearer's body located adjacent the panel and the panel is configured to hold at least one coolant, the at least one coolant is non-removable with respect to the at least one panel, the panel configured to prevent the coolant from coming into contact with a wearer's skin, the panel comprising at least two layers, one positioned between the at least one coolant and the wearer's skin and one positioned between the at least one coolant and an exterior. Further, the at least one panel may be integral with the garment body. Still, the at least one panel may be configured to attach and detach from the garment body and may be repositioned over a garment surface. Still again, the at least one panel may be placed on a garment outer surface. Moreover, the at least one panel may be placed on a garment inner surface. Yet again, the panel may comprise at least three layers with the third layer comprising at least one shielding layer, at least one tactile fabric, or at least one shielding layer. Again still, the at least one panel may be sandwiched between an upper three layer construct and a lower three layer construct wherein each three layer construct comprises at least one first shielding layer, at least one second shielding layer, and at least one tactical fabric layer. Further again, the upper three layer construct may include an outermost shielding layer. Moreover, the coolant may be configured to be flexible. Again yet, the garment body may configured as pair of pants. Still the garment body may be configured as a shirt. Again further, the garment body may be configured as a hat. Still further, the garment body may be configured as a sock. Additionally, the garment body may be configured to appear as dress clothing and lack an appearance of workout clothing and/or be configured to look like undergarments such as underwear.

In a further embodiment, a method for providing cooling treatment is provided. The method may include placing a garment in a cooling unit, the garment may comprise a garment body, at least one panel affixed to the garment body, wherein the at least one panel is configured to conform to at least one part of a wearer's body located adjacent the at least one panel and the at least one panel is configured to hold at least one coolant, the at least one panel configured to prevent the coolant from coming into contact with a wearer's skin, the at least one panel comprising at least two layers, one positioned between the at least one coolant and the wearer's skin and one positioned between the at least one coolant and an exterior, removing the garment from the cooling unit, and having a wearer don the garment to place the at least one panel adjacent to an area of a wearer's body to provide cooling treatment. Further, the garment may be removed from the cooling unit after a prescribed time. Still, the garment may be removed from the cooling unit once a predetermined temperature is reached. Further yet, the at least one panel may be repositioned on the garment to cool another area of the wearer's body. Even further, the garment may be returned to the cooling unit after a prescribed time limit. Still again further, the garment may be returned to the cooling unit after the at least one panel reaches a predetermined temperature.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows one embodiment of a cooling garment of the current disclosure.

FIG. 2 shows a multiple layer stack configuration surrounding a coolant of the current disclosure.

FIG. 3 shows one embodiment of a coolant pack of the current disclosure.

FIG. 4 shows an alternate view of FIG. 1.

FIG. 5 shows a further alternate view of FIG. 1.

FIG. 6 shows a short pant configuration cooling garment of the current disclosure.

FIG. 7 shows a multiple layer stack configuration of the current disclosure used with the short pant configuration.

FIG. 8 shows a coolant pack of the current disclosure used with the short pant configuration.

FIG. 9 shows an alternate view of FIG. 6.

FIG. 10 shows a further alternate view of FIG. 7.

FIG. 11 shows a short configuration cooling garment of the current disclosure.

FIG. 12 shows a multiple layer stack configuration of the current disclosure used with short style cooling garment.

FIG. 13 shows a coolant pack of the current disclosure used with a short style cooling garment.

FIG. 14 shows an alternate view of FIG. 11.

FIG. 15 shows a further alternate view of FIG. 11.

FIG. 16A shows an undergarment configuration of a cooling garment of the current disclosure.

FIG. 16B shows a rear view of FIG. 16A.

FIG. 17A shows a shirt configuration of a cooling garment of the current disclosure.

FIG. 17B shows a rear view of FIG. 17A.

FIG. 18A shows a photograph of a causal wear cooling garment embodiment of the current disclosure.

FIG. 18B shows a rear view of FIG. 18A.

FIG. 19 shows a sock configuration of a cooling garment of the current disclosure.

FIG. 20 shows an alternate view of the sock configuration of FIG. 19.

FIG. 21 shows a glove configuration of a cooling garment of the current disclosure.

FIG. 22 shows a hat configuration of a cooling garment of the current disclosure.

FIG. 23 shows a sports bra configuration of a cooling garment of the current disclosure.

FIG. 24 shows a rear view of the sports bra configuration of FIG. 23.

FIG. 25 shows an alternate rear configuration of the sports bra configuration with differing panel placement to provide locale specific cooling without covering substantially all of the wearer's upper back.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, sub arachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the cold therapy garments described herein can be presented as a combination kit. As used herein, the terms “combination kit” or “kit of parts” refers to the garments and any additional components that are used to package, sell, market, deliver, and/or administer cold therapy. Such additional components include, but are not limited to, packaging, coolant, cold packs, blister packages, and the like. When one or more of the garments, cooling packs, etc., described herein or a combination thereof (e.g., agent(s)) contained in the kit are administered simultaneously, the combination kit can contain the garment and coolant. When the garment and coolant described herein or a combination thereof and/or kit components are not administered simultaneously, the combination kit can contain each agent or other component in separate combinations. The separate kit components can be contained in a single package or in separate packages within the kit.

In some embodiments, the combination kit also includes instructions printed on or otherwise contained in a tangible medium of expression. The instructions can provide information regarding the content of the package, safety information regarding the content of the compounds and formulations (e.g., coolant formulations, cloth configurations, etc.), information regarding the indications for use, and/or recommended treatment regimen(s) for the compound(s) contained therein. In some embodiments, the instructions can provide directions and protocols for administering the cold therapy garments described herein to a subject in need thereof. In some embodiments, the instructions can provide one or more embodiments of the methods for administration of cold therapy methods described in greater detail elsewhere herein.

The present disclosure provides casual and comfortable clothing fused with icepacks that may be worn while moving. Shorts, pants, leggings, shirts, and underwear may be provided with thin and durable ice packs in key pain areas that are sewn into the garment and cannot be removed. Alternatively, coolant may be permanent affixed within a cloth lined, waterproof pouch and removably attached to and reaffixed/repositioned over the garment surface as icing needs change from time to time. The present disclosure allows mobility during cold therapy (i.e., icing) utilizing comfortable materials, encapsulating ice packs with seams to minimize leaks, and prevents over-icing.

Having the ice pack positioned within seams and attached to the garment allows for fluid movement by the user. The ice pack becomes part of the garment. Not removing the pack lends to a more comfortable experience, the pack will not bunch or lump, such as might occur when continually removing and reinserting a pack into a cloth packet or pouch. The lump effect occurs in current options due to attempting to force a large ice pack into a small pocket on the garment. This mismatch (large pack/small pocket) compromises the integrity of the ice pack and garment. Furthermore, the pack can easily slip out of the pocket. Properly measuring around the ice pack and completely or substantially sealing the pack within a seamed enclosure ensures no slipping, bunching, and lumping pack. The current disclosure's garments allow the pack lie flat and stay in place allowing the ice pack to operate with the full functionality intended to cool an ache, pain, or injury.

The current disclosure encloses ice packs into casual clothing, allowing people to relieve pain while being free to move. There is no complicated pack removal. The inventive fusion of clothing and ice pack changes the process of icing, supporting better circulation, and may comprise anti-leaking icepacks made of top-quality fabrics and materials as well as thinner packs to combat injury from over icing. Injuries can include nerve damage, tissue damage, and injuring areas of poor circulation. Frostbite can occur in minutes. Oxygen is critical to overall tissue health. When frostbite occurs ice crystals form in skin cells and slows blood flow depriving the cells of oxygen. Further, ice burns can cause permanent damage to a wearer's skin and underlying tissues. This disclosure provides a better way to provide cooling relief to people suffering from back, shoulder, ankle, hip joint pain as well as provide icing relief for post-surgery scaring, vaginal and cesarean delivery post-partum relief. The garments disclosed herein streamline the process of at-home cold therapy, making icing joint pain, aches, or scaring comfortable, mobile, and provide exceptional relief. Pain sufferers and physically intensive workers will not have to stop what they are doing to remove (or replace) an ice pack.

Broadly, an embodiment of the present disclosure provides a wearable cooling device comprising a garment with ice packs encapsulated therein. Generally, strong plastic bags or other suitable containers may encapsulate the coolant or ice packs. The plastic bag may be filled with a thin layer of gel or silicone beads and heat-sealed or sealed with adhesive. The ice pack may then be inserted between layered fabrics, foil fabrics, moisture resistant fabric, platinum-catalyzed silicone rubber and secured to the article of clothing with synthetic thread or heat sealed to the garment. For example, the garment, a fabric layer, the plastic/silicone bag/container with gel or silicone bead filling, a layer of moisture resistant fabric, and a layer of fabric or silicone sheet for finishing are layered and the thread or heat seal is used to weave the layers together and form a seam.

Generally, fabric thread tightly weaves the icepacks, layers of fabric, and/or platinum-catalyzed silicone rubber to seal and secure it in the garment. The sealed coolant or ice packs may be strategically placed to target and relieve pain in several areas of the body, including but not limited to: left and/or right shoulder, upper back under the shoulder blades, lower back and tailbone, outer left hip and/or right hip, inner left hip and/or right hip, left and/or right knee, lower abdomen, ankle, ball/heal of foot, head, hands, chest, under breast/ribcage, neck and groin. While these body locations are specifically delineated, the current disclosure may be used over the entirety of the wearer's body and same should be considered fully disclosed herein.

In some embodiments, a silicone overlay may be provided. Depending on the garment, the icepack may be overlaid with several layers of Lycra®, cotton jersey-Lycra® blend, tactical fabrics, foil fabrics, nylon, polyester, paper or platinum-catalyzed silicone rubber to prevent ice pack leaking.

The ice pack garment may be a garment suitable for extremities like a hat for the head, a glove for the hand, or socks for the ankles and feet. In some embodiments, the ice packs may be inserted and enclosed through seams for a form-fitting cooling tactical shirt. The present disclosure provides thinner cooling packs than currently available tactical ice pack clothing use. The garment is sewn together by first allowing an inch allotment on all sides to our customized thin ice packs. The ice pack customization includes a custom width, height, and length to accommodate the body part being covered. Thus, specific ice packs may be tailor made, literally, for a wearer based on their body configuration rather than sold as a “one-size-fits-all-but-fits-no-one” type of garment typically found in commerce. For example the tactical shirt may be created by taking our custom ice packs and covering both pectoral muscles and the ribcage. The layers of fabric may include tougher fabrics for tactical use. The custom ice pack for the back may be optional, indeed, placements shown herein should not be considered static, but able to be tailored to a wearer's particular physic. All the garments have a similar assembly: at least one custom icepack shaped specifically to mold to the desired body part, protective fabric layered to reduce ice pack leakage or icing injury, and finishing fabric for design. The first iteration of these garments will go in to a refrigerator or cooling unit that reaches temperatures below 50° Fahrenheit (10° C.). Recommended cooling time may be up to one hour before ready to be worn. Once at a desired cooling temperature, the garment may be worn like any traditional legging, pant, shirt, hat, glove, underwear (panty/bra), or sock. Indeed, in other embodiments, the garments may eliminate the need for refrigeration for cooling. The garments may employ insta-cool technology (ice packs that use a forceful strike to initiate the cooling process) or wired technology that connects to an application on the user's smartphone to control the cooling or heating of the icing unit on the garment via circulating coolant or heated fluids, inducing a heating coil, circulating warmed or cooled air, etc.

A method of using the disclosure may include the following. The inventive hybrid ice pack garment may be placed in a cooling unit such as a freezer, refrigerator, cooling bath, cold air flow, etc., for example for a predetermined amount of time or until a predetermined temperature is reached, such as for purposes of example only, a minimum of thirty minutes, to activate the cooling properties of the coolant, which may be a cooling gel, silicone beads, etc. The ideal temperature may be about 39 degrees Fahrenheit but depends on the cooling needs of the user. Ranges of temperatures are considered from 0 to 40 degrees Fahrenheit including subranges such as from 5 to 35, 10 to 30, 15 to 25, and specific temperatures such as 10, 15, 20, 30, 35, etc. The user may then remove the garment from the cooling unit and don the garment. The garment may be put on the body like traditional shirts, shorts, leggings, or underwear. The coolant or ice packs will preferably be positioned against the user's painful areas. The user may choose to remain stationary during the cold therapy or participate in activities that require mobility. The garment may be returned to the cooling unit after a preset time limit or after the coolant reaches a predetermined temperature at which further cooling is necessary to help provide icing therapy.

In one embodiment of the present disclosure, the hybrid garment may be used for the medical industry to replace the ice pack typically given to surgery patients with more cost-effective fabrics that may be produced in bulk. The disclosure may also be used as an affordable cooling device for underdeveloped global regions afflicted by oppressive heat (e.g., above 100 degrees Fahrenheit). The disclosure may be used for tactical purposes and a stronger embodiment may be provided with longer cooling times for use by the military, firefighters, police, and construction workers who work in hot conditions. Longer cooling therapy times may be sustained by utilizing foil fabrics to prolong the cooling effect of an ice pack longer. For tactical wear embodiments, the layering of fabric will rely on conductive cooling. The foil material becomes conductive once contact is made with the chilled ice pack. The foil material holds the cold temperature longer versus garments without the foil layering.

The method of manufacture is not particularly limited. Lycra®, cotton jersey Lycra® blend, or tactical, or foil fabrics and fabric threads may be woven to directly produce different types of clothing. Alternatively, the Lycra®, cotton jersey-Lycra® blend, or tactical/foil fabric may be sewn from patterns into traditional articles of clothing (shirts, shorts, pants, leggings, or underwear) with fabric thread or heat sealing as known to those of skill in the art. Casual clothing (shirts, shorts, leggings, and underwear) may be produced by an individual with a sewing apparatus or may be outsourced to a commercial garment manufacturer. The coolant may be made by an individual or outsourced to a cold therapy manufacturer. The molded silicone overlay may be provided by a silicone mold manufacturer. The sealing and fusion of the ice packs to casual clothing may also be done by an individual with a sewing apparatus or outsource the process to a commercial garment manufacturer. Once the garment and the coolant is made, the coolant may be sewn into the garment and overlaid with fabric or silicone. Silicone ice packs are an option for use as well as ice packs sealed in a thick plastic layer. We may have users who prefer silicone ice packs to plastic as a more sustainable, flexible, and comfortable material to hold the cooling gel or silicone beads. Our custom silicone ice packs are molded by an outsourced silicone manufacturer. Once we receive the pack it is assembled and affixed, similarly, to our garments by layering fabric around the silicone pack and sealing it to the garment with synthetic thread or heat seal. Clothing tags may be attached to clarify garment sizing, care, cleaning instructions, how to use the garment, and company branding.

The materials of manufacture are not particularly limited and may include a combination of materials selected from the group consisting of: Lycra® fabric; cotton jersey and Lycra® blend fabric; tactical fabric; foil fabric; cooling gel or silicone beads; plastic bag (plastic casing); pre-shaped molded platinum-catalyzed silicone rubber; fabric thread (natural or synthetic); and adhesive or sealant.

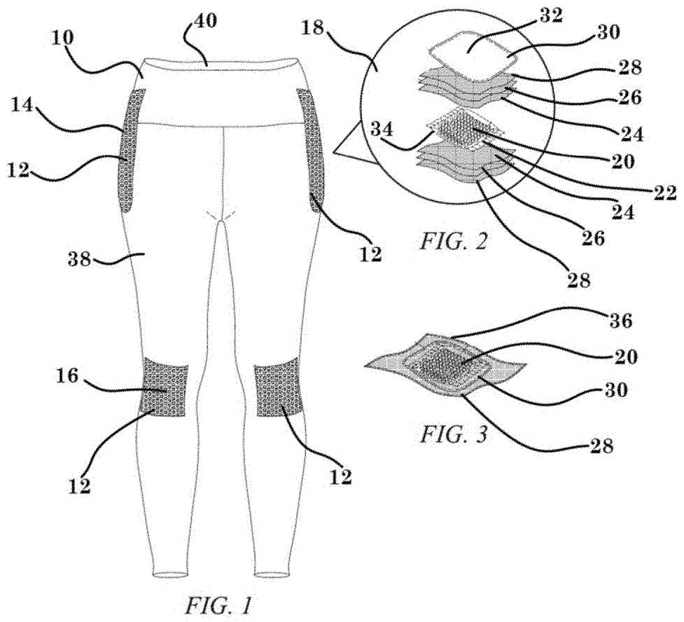

Referring now to the Figures. FIG. 1 shows a cooling garment 10, in this embodiment shown as a pair of leggings. Cooling garment 10 may include panels 12, which are shaped to contour to parts of the wearer's body such as hips, knees, elbows, neck, biceps, pectorals, hands, joints, etc. As FIG. 1 shows, panels 12 are configured to shape or conform to the wearer's body located adjacent the panel, such as hips 14 and knees 16. This improves on static or rigid ice packs that simply lay atop one, small portion of the wearer's skin, such as a rectangular ice pack that would only contact a small portion of the wearer while the rigid nature of the pack keeps the remainder of the icing surface from contacting the wearer. For this disclosure, panels 12 are designed to surround and hold a flexible coolant or ice pack. Herein, “flexible” means capable of bending easily without breaking, as well as assuming different contorted shapes and being able to return to the original shape without permanently altering the shape of the coolant or ice pack.

In one instance, see FIGS. 2 and 3, a panel of the current disclosure may be configured to possess a plurality of layers, with at least one of the layers positioned between the at least one cooling pack and the wearer's skin and one layer positioned between the at least one cooling pack and an exterior of the garment or an exterior environment surrounding the wearer. Panels 12 may comprise a plurality of layers 18. These layers may include a thin layer of coolant 20, which may be in the form of a cooling gel, silicone beads, cherry pits, water, rubbing alcohol, ammonium chloride, calcium chloride, urea, magnesium sulfate, sodium acetate, ammonium nitrate, calcium ammonium chlorides, solid silicone, conductive coils, wire, polymer refrigerant, conductive materials, plastic tubing, microchips, aluminum foil, solid aluminum beads, aluminum disc, glass beads, Bluetooth technologies, sand, plastic, plastic beads, salts, air, refrigerant chemicals, propylene glycol, dye, hydroxyethyl cellulose, vinyl-coated silica gel, sodium acetate, or any combination of the aforementioned components intended as possible fillings encapsulated within a strong polymeric pouch 22, which may comprise nylon, polyurethane, silicone, linear low density polyethylene, low density polyethylene, high density polyethylene, or latex sandwiched between a first shielding layer 24, which may be a layer of Lycra®, a second shielding layer 24, which may be a cotton jersey-Lycra® blend, and a tactical fabric 26 tactical fabrics are made for high impact, intense use, long-term wear, and resistance/combating/repelling dangerous elements like: fire, harsh/dangerous chemicals, extreme force, or extreme weather conditions. Ideal tactical fabrics and tactical fabric coatings (coatings are types of treatments that can be added to fabrics to enhance it and serve a specific function for the garment) include: CORDURA® fabrics, Mil-Spec nylons, cottons, polyesters, stretch wovens, ripstop/ripstop construction (a type of fabric weaving that makes the fabric more durable), nylon cotton ripstop, poly cotton ripstop, poly cotton twill, canvas, ceramic dots coating, heatproof fabric, flame-resistant fabric, flame-retardant fabric, GORE-TEX® moisture wicking coating, No-Melt/No-Drip fabrics, LYCRA®, lyocell fabric, Nyco fabric, nyco extreme fabric, merino wool, polyamides fabrics and coatings, PolyCotton fabric, PIRMALOFT® fabric, down, SCHOELLER®-dynamic stretch fabric, VELCRO®, woven fabrics, 37.5™ technology materials, yarn, and yarn, outermost shielding layer 30 may be a layer of platinum-catalyzed silicone rubber or any of the materials mentioned above as well as foil fabric on outer surface 32 of panel 12. This multilayer or stacked combination may be repeated on adjacent the wearer facing surface 34 of panel 12 to provide a second first shielding layer 24, second shielding layer 26, and tactile fabric layer 28 between the wearer's skin and coolant 20. FIG. 3 shows a partial see-through view of panel 12, which may be stitched together with thread 36 and woven onto garment outer surface 38 or garment inner surface 40, see FIG. 1, or sewn between layers forming cooling garment 10.

The layering of the fabrics around the ice pack serves the purpose of ensuring anti-leaking, prolonged cooling, and matching the aesthetic of the rest of the garment. The first layer and most immediate, adjacent to the ice pack is the moisture resistant fabric. Its purpose is to stop any leaking of the gel coolant, silicone bead, or any other coolant inside of the plastic pouch by acting as a barrier and effectively another “pouch” when fused together with synthetic thread and then to the garment. The next layer is an optional foil fabric to prolong cooling by acting as a conduit by absorbing the cool temperature of the ice pack and maintaining that temperature longer. Next is a cotton or terry fabric to create a soft pliable layer to protect the skin and prevent frostbite, nerve, or muscle damage by creating a barrier between the ice pack and adsorbing intensity of the cold temperature. The final layer is a fabric match to the aesthetic of the garment (like cotton or Lycra) or to provide branding opportunities. The number of fabric layers can vary per garment or based on the purpose of garment. The layers are equal on both sides and the variation is at least four (sheet of fabric) on top, four on bottom and up to any number combination needed to effectively produce the garment and fused by synthetic thread.

The panels 12 of the current disclosure may be permanently sewn into the garment and considered to be a part of the functionality and utility of the garment. This permanence of the panel allows for anti-slipping of the ice pack, free movement of the user, and ensuring the ice pack lies flat for better construction, manufacturing, and comfort for the user. Different customization or variations of the fusion of the panels to the garment may be produced upon request or for the purpose of iteration change or product development. The panels may be: pockets or fused on three sides and an ice pack can be inserted or removed, or said pocket can be sealed and fused to the garment on three sides with synthetic thread and sealed with a zipper on the forth side allowing the ice pack to be removable and replaceable. Zippers can be fused to the garment in any variation of the four-sided panel 12. All four sides can be zipped, three sides zipped, two sides zipped, etc., each allowing for a removable ice pack on the exterior of the garment. The panel may be sealed on all four sides but be moved and repositioned fused to the exterior of the garment via VELCRO®, snap buttons, snap fasteners, fabric adhesives, fabric adhesive sheet, fabric adhesive tape, flat buttons, shank button, stud button, or toggle button.

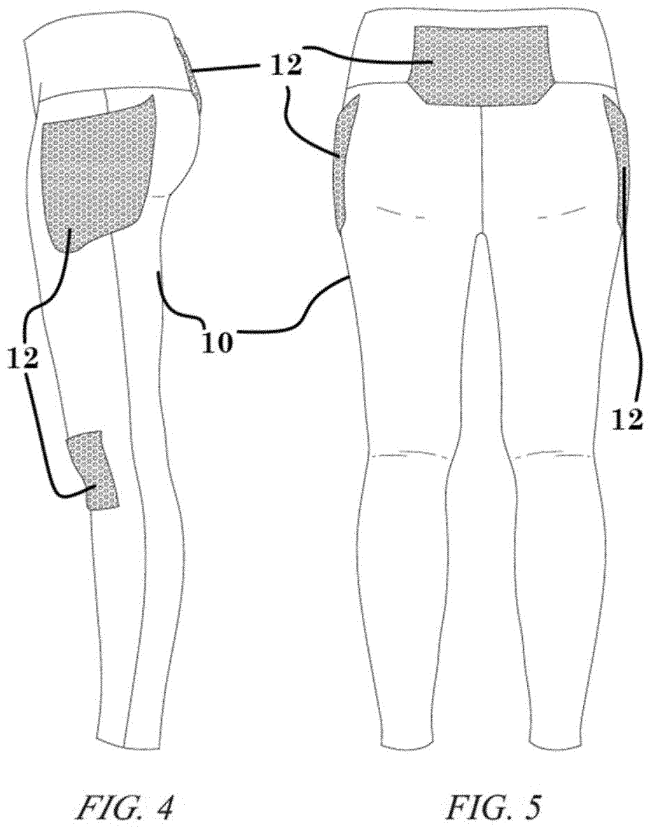

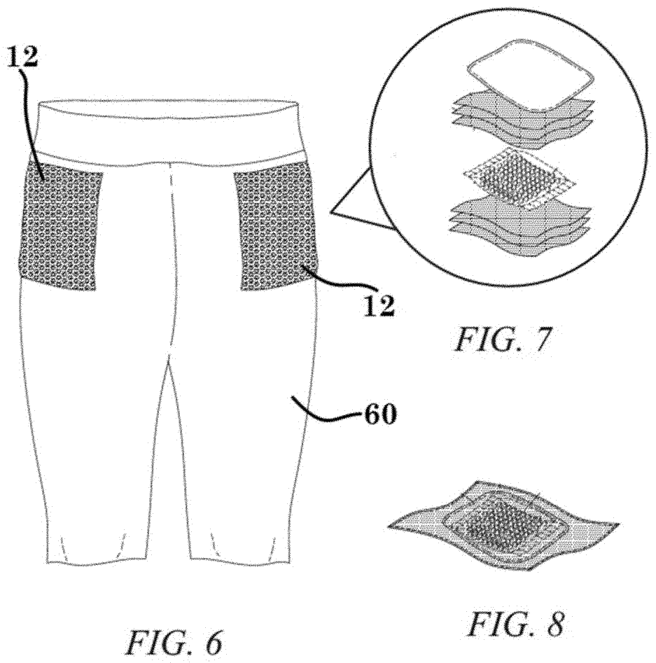

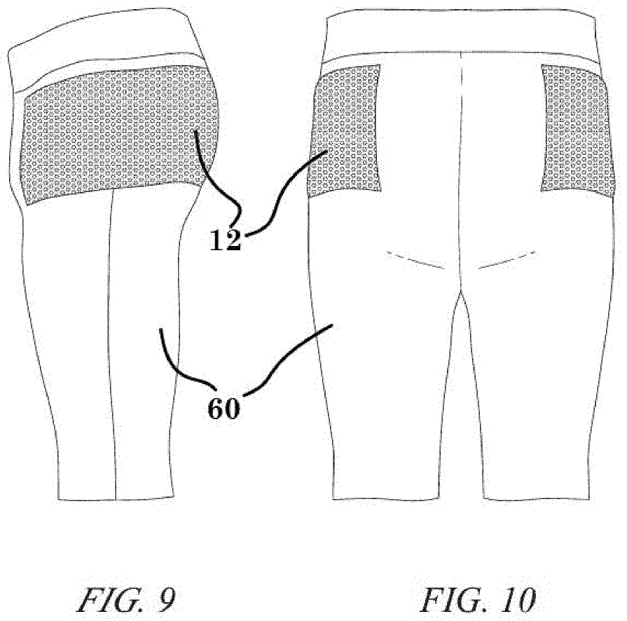

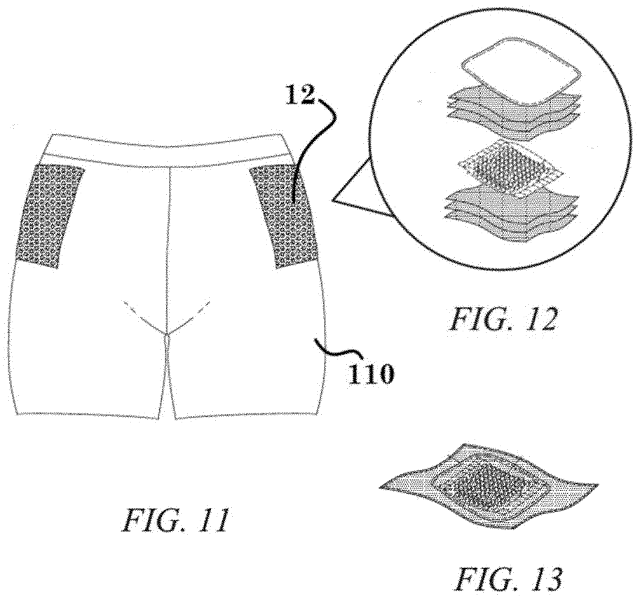

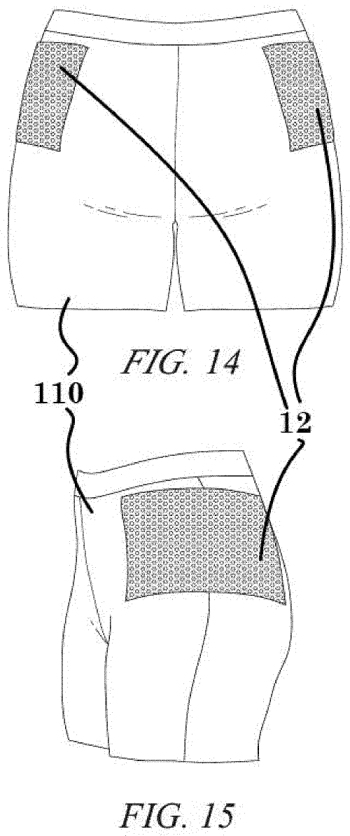

FIG. 4 shows back and side views of garment 10, as well as possible further placement areas for panels 12. While particular locations on garment 10 and garment outer layer 38 are shown, placement is not so limited and the disclosure should be considered to cover placing panels 12 anywhere on garment 10 either on the inner surface or outer surface. FIGS. 6-8 show a short pant configuration 60 of the cooling garment with alternate placemen of panels 12, panels 12 shown in FIG. 7 share the configuration of FIG. 2 and FIG. 8 shares the configuration of FIG. 3. FIGS. 9 and 10 shows alternate views of FIG. 8. FIG. 11 shows short configuration 110 of one embodiment of a cooling garment of the current disclosure. Panel 12 shown in FIG. 12 has the configuration of FIG. 2 and FIG. 13 has the configuration of FIG. 3. FIGS. 14 and 15 show alternate views of short configuration 110.

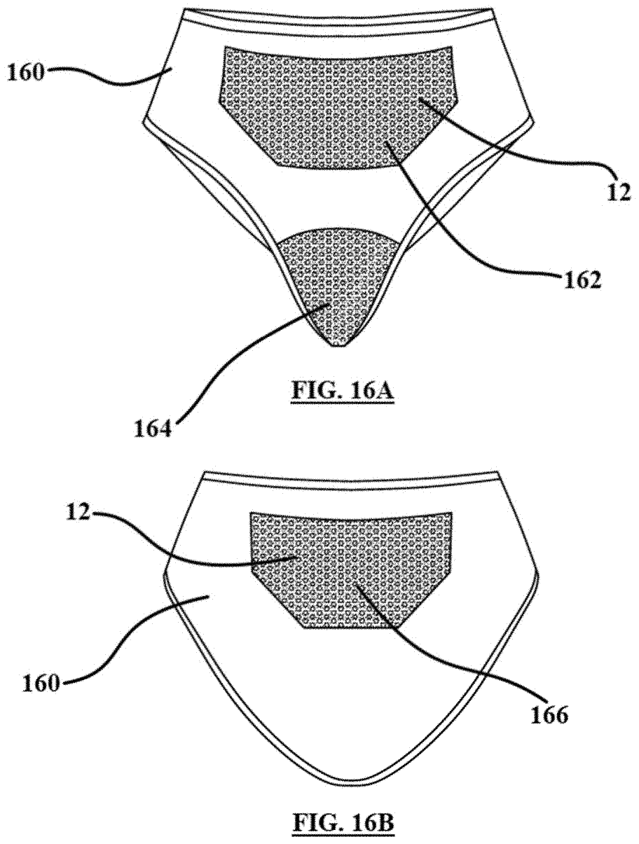

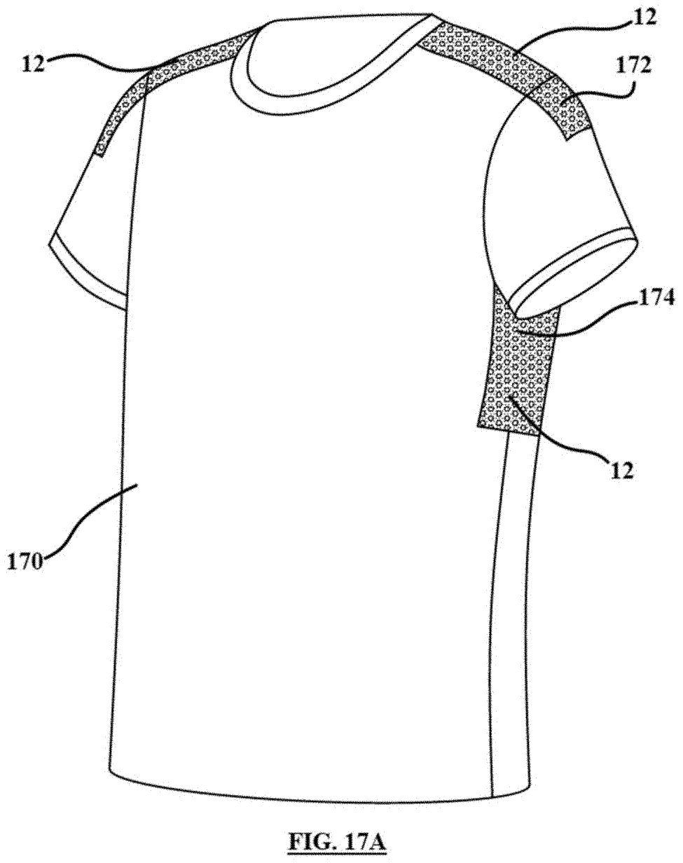

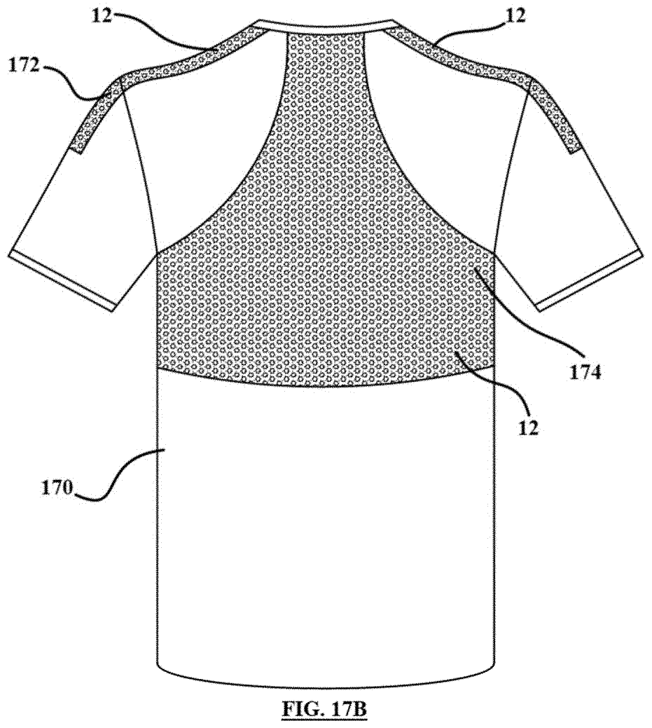

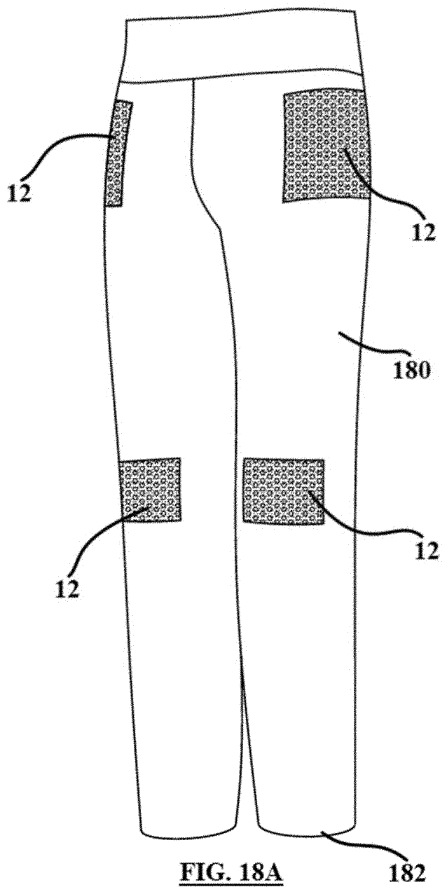

FIGS. 16A and 16B show an undergarment configuration 160, such as men or women's underwear, wherein panels 12 may be placed at the abdomen 162, crotch 164, lower back 166, etc., of the wearer to provide cooling in these areas that are not normally suitable for ice packs or bags of ice. FIGS. 17A and 17B show a shirt embodiment 170 of a cooling garment of the current disclosure with panels 12 disposed on shoulders 172 and underarms 174 of the garment.

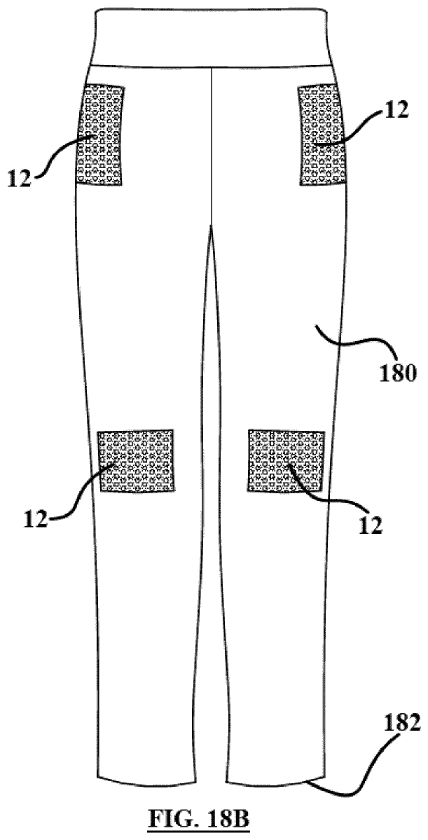

FIGS. 18A and 18B show a casual wear 180 embodiment of the current disclosure wherein the cooling garment may be fashioned not to appear like workout clothing but to instead appear as work or casual wear clothing. This allows the wearer to not only be able cool desired parts of their body but to be able to do so in social or work settings without appearing underdressed or inappropriately dressed for the location where they are present. While panels 12 may be formed as differently colored from garment 180, panels 12 may be made the same color as the garment to further lessen their noticeability. Indeed, adding straight cut cuffs 182 to the cooling garment and changing panels 12 to share the color of the cooling garment would serve to render the garment indistinguishable from everyday attire, if the wearer did not want others to know they were using the cooling garment or coolant. However, in an alternate embodiment panels 12 may be colored so that when worn on the outside of a garment, one may distinctly see the panels in contrast to the underlying garment. Indeed, indicia such as advertising, logos, pictures, etc., may be used to decorate panels 12 to add flair to clothing. Further, panels 12 may come in a wide variety of shapes, such as those shown in the FIGS., square, rectangular, oval, circular, irregularly shaped, precise shapes such as stars, polygons, irregular shapes such as the “blots” appearing in a camouflage pattern, etc. Indeed, while some embodiments show panels 12 in juxtaposed or opposing locations on a wearer's physique, this is not required and placement may be sporadic or specifically chosen by the wearer.

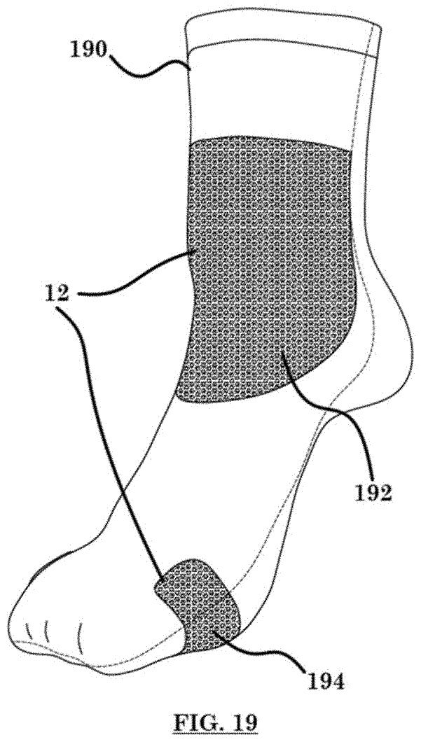

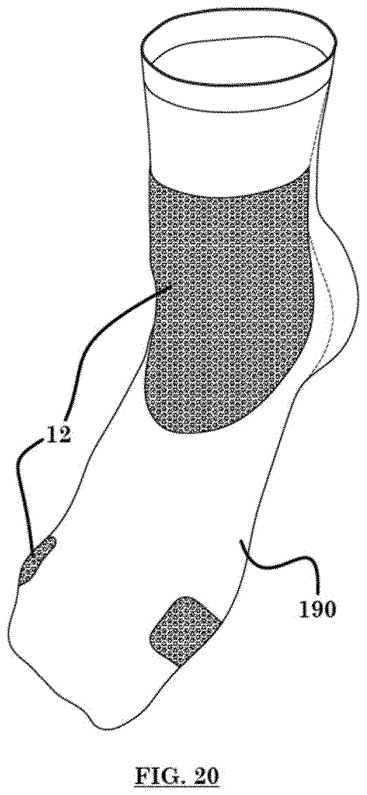

FIG. 19 shows a sock configuration 190 embodiment of a cooling garment of the current disclosure. As FIG. 19 shows, panels 12 may be placed at bridge 192 and at the ball 194 of a foot to provide enhanced cooling while securing coolant in place, even during walking, running or other movement. FIG. 20 shows an alternate view of FIG. 19.

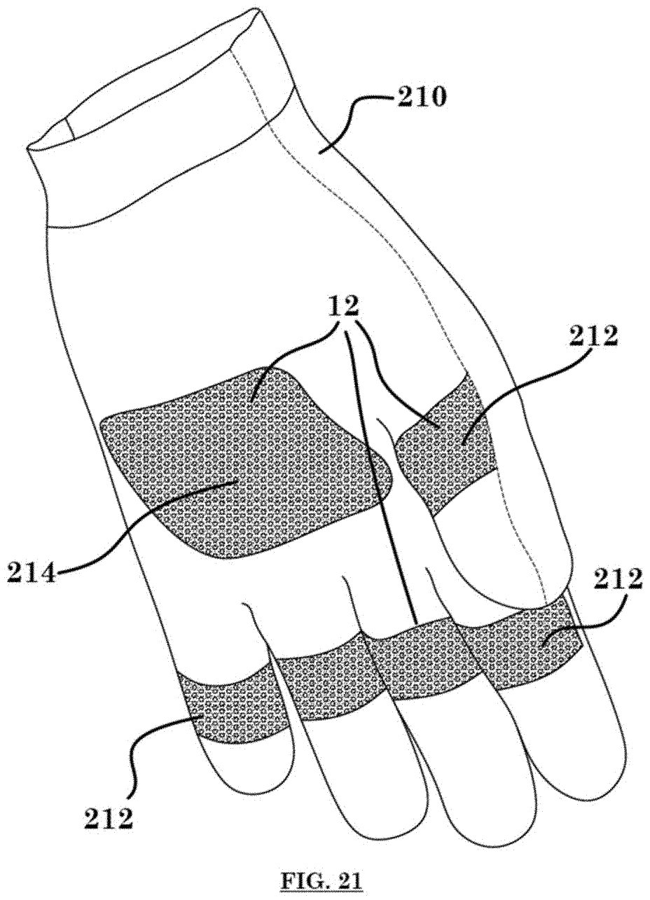

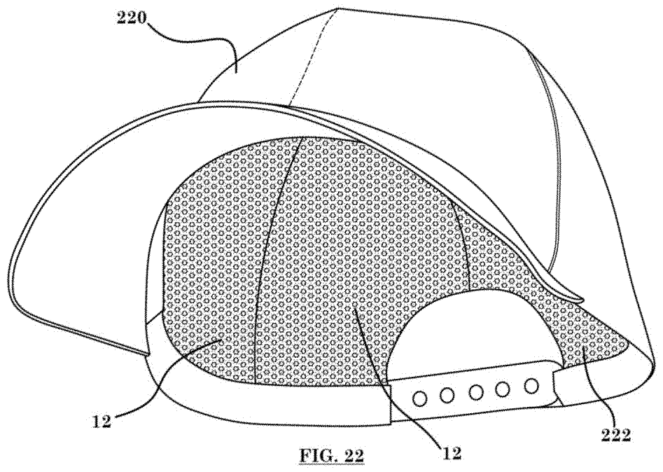

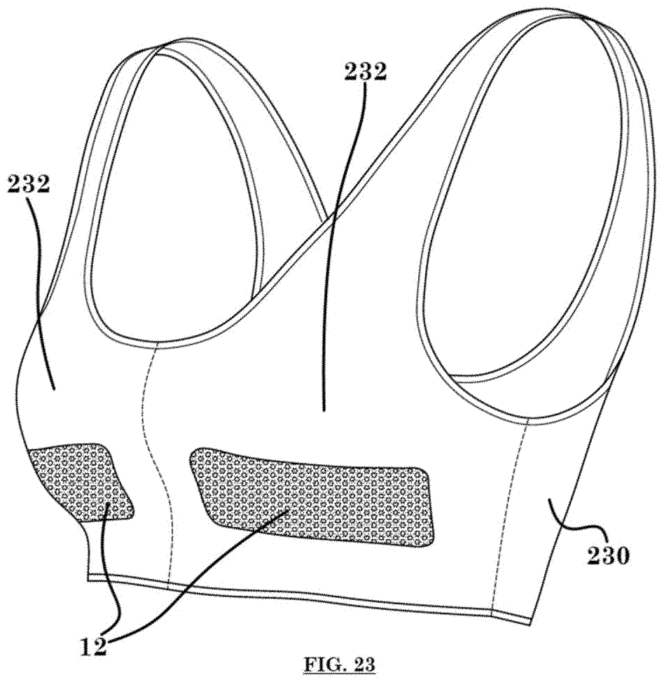

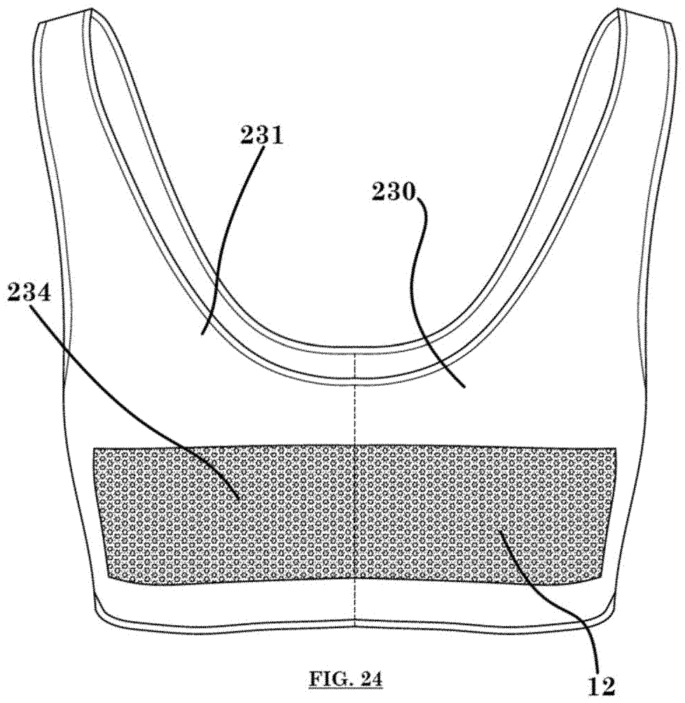

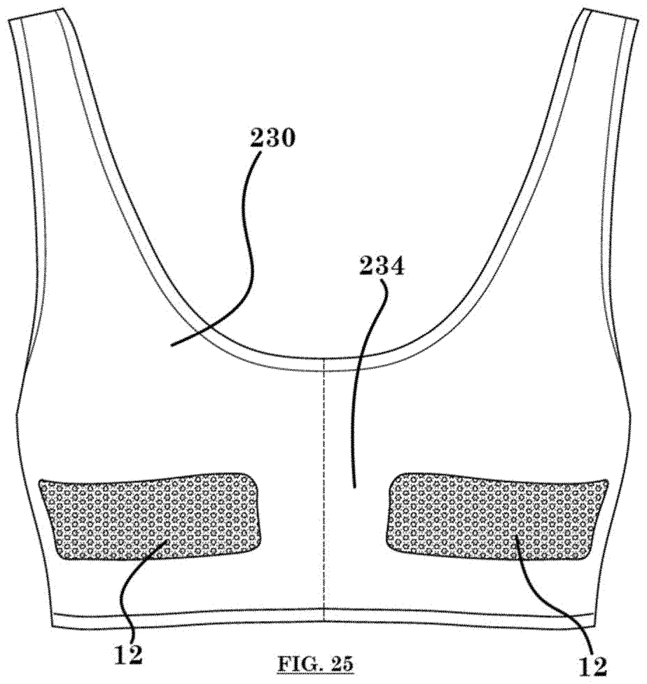

FIG. 21 shows a glove configuration 210 cooling garment of the current disclosure wherein panels 12 may be located on finger joints 212 and palm 214 of the wearer. FIG. 22 shows a hat configuration 220 cooling garment wherein panels 12 may cover the entirety of hat interior 222, or may be positioned as only in part of hat interior 222. FIG. 23 shows sports bra configuration 230 wherein panels 12 may be place on or under breasts 232 to provide cooling. FIG. 24 shows a rear view of sports bra configuration 230 where panel 12 covers substantially all of the wearer's upper back 234. FIG. 25 shows an alternate rear configuration of sports bra configuration 230 wherein multiple panels 12 are provided to provide locale specific cooling without covering substantially all of the wearer's upper back. 234.

For purposes of example only and not intended to be limiting cooling hat garments may be used by placing on the user's head. The cooling pack surrounds the cranium providing immediate cooling relief. The hat is best used for migraines/headaches, when working/playing/out in hot weather conditions for prolonged periods, or as quick way to cool down after a period of exertion. It may also be used as a post-surgical relief device/garment. Meanwhile, cooling glove garments may be used to provide cooling relief to the hand. This includes digits, wrist, fingers, thumb, palm, and knuckles. The ideal usage for this glove is after gripping heavy items, after gripping hot items, as a solution to burns on the hand, or as a way to sooth sprains or aches in the joints of the hand: the wrist and knuckles. Can be used as a post-surgical relief device/garment. The cooling shirt embodiments may be used for soothing shoulder sprains and aches, as well as upper back pain or neck pain. The shirt may be best used to sooth pain, and provide cooling relief when working/playing/being in hot weather conditions for prolonged periods, or during leisurely activities.

The various long pant/shorts shaped cooling garments, whether athletic or casual (street wear) in appearance, may provide cooling relief in specific points of the user's lower body, specifically, the lower back, outer hip, inner hip, and left and right knees. The leggings/pants/shorts can be used during bouts of exertion, before, during, and post athletic activities, or during leisurely activities. The user is still able to do task and ice pain to the lower body. Can be used as a post-surgical relief device/garment.

Undergarment embodiments, such as underwear bras, may be used as a cooling device for relief to the breast, upper back lower abdomen, lower back, or genitalia. The cooling underwear is ideal for postpartum recovery. The ice packs covers a cesarean surgery incision or provides cooling to the vaginal area post vaginal birth. The underwear also soothes lower back pain. The underwear is a post-surgical option for transgender or genital surgery. The underwear may also be a holistic option to sooth hemorrhoid pain. The underwear and bra may also act as a fast acting cooling relief for menopausal women suffering from hot flashes. The bra is used to provide back pain relief for women who are well-endowed and suffer from back pain. The bra also is a post-surgical relief garment after breast augmentation surgery.

Cooling sock garments may relieve pain from an injured or painful ankle. The sock also provides relief to the balls of the feet. Pain to the balls of the foot may source from wearing heeled shoes or prolonged athletic activity causing trauma or pain to the foot. The sock garments may also be used as a post-surgical relief device/garment.

It should be understood, of course, that the foregoing relates to exemplary embodiments of the disclosure and that modifications may be made without departing from the spirit and scope of the disclosure as set forth in the following claims.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A cooling garment comprising:
a garment body;
at least one panel affixed to the garment body, wherein the panel is configured to conform to at least one part of a wearer's body located adjacent the at least one panel and the at least one panel is configured to hold at least one coolant;
wherein the at least one coolant is non-removable with respect to the at least one panel;
the panel configured to prevent the coolant from coming into contact with a wearer's skin; and
the panel comprising at least two layers, one positioned between the at least one coolant and the wearer's skin and one positioned between the at least one coolant and an exterior.

2. The cooling garment of claim 1, wherein the at least one panel is integral with the garment body.

3. The cooling garment of claim 1, wherein the at least one panel is configured to attach and detach from the garment body and may be repositioned over a garment surface.

4. The cooling garment of claim 1, wherein the at least one panel is placed on a garment outer surface.

5. The cooling garment of claim 1, wherein the at least one panel is placed on a garment inner surface.

6. The cooling garment of claim 1, wherein the panel comprises at least three layers with the third layer comprising at least one shielding layer, at least one tactile fabric, or at least one shielding layer.

7. The cooling garment of claim 6, wherein the at least one panel is sandwiched between an upper three layer construct and a lower three layer construct wherein each three layer construct comprises at least one first shielding layer, at least one second shielding layer, and at least one tactical fabric layer.

8. The cooling garment of claim 7, wherein the upper three layer construct includes an outermost shielding layer.

9. The cooling garment of claim 1, wherein the coolant is configured to be flexible.

10. The cooling garment of claim 1, wherein the garment body is configured as pair of pants.

11. The cooling garment of claim 1, wherein the garment body is configured as a shirt.

12. The cooling garment of claim 1, wherein the garment body is configured as a hat.

13. The cooling garment of claim 1, wherein the garment body is configured as a sock.

14. The cooling garment of claim 1, wherein the garment body is configured to appear as dress clothing and lack an appearance of workout clothing.

15. A method for providing cooling treatment comprising:
placing a garment in a cooling unit;
wherein the garment comprises:
a garment body;
at least one panel affixed to the garment body, wherein the at least one panel is configured to conform to at least one part of a wearer's body located adjacent the at least one panel and the at least one panel is configured to hold at least one coolant;
the at least one panel configured to prevent the coolant from coming into contact with a wearer's skin;
the at least one panel comprising at least two layers, one positioned between the at least one coolant and the wearer's skin and one positioned between the at least one coolant and an exterior;
removing the garment from the cooling unit; and
having a wearer don the garment to place the at least one panel adjacent to an area of a wearer's body to provide cooling treatment.

16. The method of providing cooling treatment of claim 15, wherein the garment is removed from the cooling unit after a prescribed time.

17. The method of providing cooling treatment of claim 15, wherein the garment is removed from the cooling unit once a predetermined temperature is reached.

18. The method of providing cooling treatment of claim 15, wherein the at least one panel is repositioned on the garment to cool another area of the wearer's body.

19. The method of providing cooling treatment of claim 15, wherein the garment is returned to the cooling unit after a prescribed time limit.

20. The method of providing cooling treatment of claim 15, wherein the garment is returned to the cooling unit after the at least one panel reaches a predetermined temperature.
